(19) United States
(12) Patent Application Publication
(10) Pub. No.: US 2012/0141989 A1
(43) Pub. Date: Jun. 7, 2012

(54) KIT AND METHOD FOR RAPIDLY DETECTING A TARGET NUCLEIC ACID FRAGMENT

(75) Inventors: TZONG-YUEH CHEN, TAINAN (TW); GWO-BIN LEE, TAINAN (TW); CHIH-HUNG WANG, TAINAN (TW); TING-YU WANG, TAINAN (TW)

(73) Assignee: NATIONAL CHENG KUNG UNIVERSITY, TAINAN CITY (TW)

(21) Appl. No.: 13/228,842

(22) Filed: Sep. 9, 2011

(30) Foreign Application Priority Data
Dec. 6, 2010 (TW) 099142290

(51) Int. Cl. C12Q 1/68; C12M 1/34

(52) U.S. Cl. 435/6.11; 435/287.2

(57) ABSTRACT

The invention provides a kit for rapidly detecting a target nucleic acid fragment comprising a magnetic bead; an inner primer pair and an outer primer pair suitable for loop-mediated isothermal amplification; and reagents for loop-mediated isothermal amplification. The invention also provides a kit for detecting a pathogen in fish, a method for rapidly detecting a target nucleic acid fragment, and a method for detecting a pathogen in fish.

### BACKGROUND OF THE INVENTION

1. Field of the Invention

The invention relates to nucleic acid detection, and more particularly to a kit and method for rapidly detecting a target nucleic acid fragment.

2. Description of the Related Art

The detection of a nucleic acid fragment is widely used in various fields and important. For example, the rapid and accurate detection of a target nucleic acid fragment can be applied for quickly diagnosing a pathogen and is benefit for early prevention.

Rapid and accurate diagnosis leading to the quarantine of aqua-culture diseases has played a crucial part in protecting fisheries. Especially, the rapid identification of infectious diseases for species with a high economic value (e.g. grouper, eel or porgy) has attracted considerable interest in recent years. However, the immune system of a fish may be cross-infected by a variety of pathogens such as viruses, bacteria, fungi or parasites in various stages of development. Hence, the development of rapid, accurate, and sensitive diagnostic platforms for the identification of pathogens have played a fundamental role in treating, controlling, or even eradicating these infectious aquaculture diseases. Traditionally, several methods including bacteriological analysis, virus isolation and culture, histopathology and an enzyme-linked immunosorbent assay (ELISA) (Adams and Thompson, 2008, Rev. Sci. Technol. 27, 197-209) have been developed for the phenotypic characterization and resulting identification of these aquaculture pathogens. For example, viral nervous necrosis is a serious viral disease in the grouper cultivation industry. Many stages in the grouper life cycle can be infected with NNV, especially in hatchery-reared larvae and juveniles (Chi et al., 2003, Dis. Aquat. Organ. 55, 221-228). The NNV has been reported to be a major cause of mortality in the larvae and juveniles of farmed marine fish throughout the world (Shieh and Chi, 2005, Dis. Aquat. Organ. 63, 53-60). The necrosis and vacuolation of central nervous tissues result in abnormal swimming behavior in the infected species, which leads to a high mortality rate in infected fishes. The infected grouper may become a carrier and an outbreak may spread quickly if the quarantine is not imposed. There is a great need for a rapid and accurate diagnostic method for the prevention and control of this disease.

Alternatively, molecular diagnosis based on polymerase chain reaction (PCR), RT-PCR, (Dhar et al., 2002, J. Virol. Methods 104, 69-82; Nishizawa et al., 1995, J. Gen. Virol. 76, 1563-1569) or quantitative real-time PCR (DallaValle et al., 2005, Vet. Microbiol. 110, 167-179) incorporated with specific primer sets for nucleic acid amplification has been demonstrated for accurate diagnosis of aquaculture diseases with a high sensitivity and specificity. The current “gold-standard method” for detection of NNV uses a conventional RT-PCR method (Nishizawa et al., 1995, J. Gen. Virol. 76, 1563-1569). The detection limit of 100-1000 copies of in vitro transcribed viral RNA in the RT-PCR assay has been demonstrated (Grotmol et al., 2000, Dis. Aquat. Organ. 39, 79-88).

However, there still exist some disadvantages, such as the need for an expensive and bulky thermal cycler, multiple and complex operating processes and low amplification efficiency (Mori et al., 2001, Biochem. Biophys. Res. Commun. 289, 150-154; Tomita et al., 2008, Nat. Protoc. 3, 877-882). Furthermore, sample pre-treatment still remains a technically demanding and time-consuming step. The quality of RNA extraction could affect the results of the RNA-virus diagnosis. A hot phenol extraction or RNA purification kits are common methods for RNA purification and separation. In addition, the requirements for PCR-based platforms are technically demanding such as the precise temperature control necessary during the thermal cycling with the temperature variation ranging from 42° C. to 95° C., which is commonly performed by costly and bulky apparatus. In addition, the lengthy and costly diagnostic processes always need to be performed by well-trained personnel and the inaccuracy of the diagnosis may be attributed to these manual operations.

Accordingly, “isothermal amplification techniques,” which allow exponential amplification of target nucleic acids at a constant and low temperature, has been developed for rapid detection of target DNA sequences (Piepenburg et al., 2006, PLoS Biol. 4, e204; Starkey et al., 2004, Dis. Aquat. Organ. 59, 93-100; Walker et al., 1994, Nucleic Acids Res. 22, 2670-2677). Among them, the loop-mediated-isothermal-amplification (LAMP) technique has attracted considerable interests as a potentially rapid, accurate, and cost-effective method for nucleic acid amplification. Specific nucleic acid sequences in the target samples can be amplified by using four designated primers with the incorporation of Bst DNA polymerase, which is capable of high strand displacement under isothermal conditions (about 60-65° C.) (Notomi et al., 2000, Nucleic Acids Res. 28, e63). Three major steps including an initial step, a cycling amplification step and an elongation step are conducted under a constant thermal condition and efficient amplification can be achieved since there is no time required for temperature ramping during the LAMP process (Nagamine et al., 2002, Mol. Cell. Probes 16, 223-229). In addition, the final amplified stem-loop DNAs consisting of cauliflower-like structures with multiple loops yields an amplification of 10⁹ copies of target DNA molecules, so that approximately a 100-fold greater sensitivity for LAMP amplification is demonstrated when compared with a conventional PCR process. As a consequence, a new diagnostic strategy incorporated the LAMP technique for fast and accurate detection of target genes has been demonstrated. For example, a LAMP-based detection of Edwardsiella tarda from infected Japanese flounder has been reported by targeting the haemolysin gene (Sayan et al., 2004, Appl. Environ. Microbiol. 70, 621-624). Another two-step RT-LAMP protocol for identification of the G-protein associated with the infectious haematopoietic necrosis virus (IHNV) in fish was also developed (Gunimaladevi et al., 2005, Arch. Virol. 150, 899-909). Despite the attractiveness of the LAMP technique, there are still some potential drawbacks in developing rapid diagnostic devices utilizing these state-of-the-art laboratory techniques. The entire nucleic acid amplification process is still costly and labor-intensive which utilizes lab-scale equipment such as pipettes and bulky thermo-heaters with a relatively large amount of bio-samples/reagents. More importantly, bio-sample pre-treatment processes prior to analysis such as DNA/RNA extraction are always required and need to be performed by experienced personnel. Furthermore, there is a high risk of contamination of bio-samples during the entire diagnostic process, which may hinder the practical applications in the field survey. Therefore, there is a great need to develop an integrated sample-to-answer system to carry out all the diagnostic processes with a high specificity and sensitivity, in an automatic manner.

### SUMMARY OF THE INVENTION

The present invention provides an integrated microfluidic LAMP system for rapidly detecting a target nucleic acid fragment.

One subject of the invention is to provide a kit for rapidly detecting a target nucleic acid fragment, the target nucleic acid fragment comprising a purification recognized fragment and an amplification specific fragment, which kit comprises:

a magnetic bead linked to an oligonucleotide being able to hybridize to the purification recognized fragment;

an inner primer pair and an outer primer pair being specific to the amplification specific fragment and suitable for loop-mediated isothermal amplification; and

reagents for loop-mediated isothermal amplification.

Another subject of the invention is to provide a kit for detecting a pathogen in fish by detecting a pathogen target nucleic acid fragment in a sample, which kit comprises the kit for rapidly detecting a target nucleic acid fragment mentioned above.

Still another subject of the invention is to provide a method for rapidly detecting a target nucleic acid fragment, the target nucleic acid fragment comprising a purification recognized fragment and an amplification specific fragment, which method comprises:

(a) purifying a nucleotide with a magnetic bead, wherein the magnetic bead linked to an oligonucleotide being able to hybridize to the purification recognized fragment;

(b) conducting a loop-mediated isothermal amplification with an inner primer pair and an outer primer pair being specific to the amplification specific fragment and the nucleotide purified in step (a), wherein the inner primer pair and the outer primer pair are suitable for loop-mediated isothermal amplification; and

(c) detecting a product of the loop-mediated isothermal amplification.

Still another subject of the invention is to provide a method for detecting a pathogen in fish by detecting a pathogen target nucleic acid fragment in a sample, which method comprises the method for rapidly detecting a target nucleic acid fragment mentioned above.

### BRIEF DESCRIPTION OF THE DRAWINGS

FIG. 1 illustrates a photograph of the integrated microfluidic LAMP system integrated with a microfluidic control module and the nucleic acid amplification module. The dimensions of the microfluidic chip are measured to be 44 mm×22 mm.

FIG. 2 illustrates the optimal conditions of the one-step RT-LAMP assay. (a) Reaction temperature optimization. Lanes 1-6 indicate the results for the RT-LAMP performed at 57° C., 59° C., 61° C., 63° C., 65° C., and 67° C., respectively. Lane L: 50-bp DNA ladders. Lane NC uses ddH₂O. (b) Hybridization temperature optimization of the RNA1-probe-conjugated magnetic beads. Lanes 1-4 represent the RT-LAMP results using the probes pre-hybridized with cDNA from NNV RNA1 at 58° C., 60° C., 63° C., and 65° C. for 30 min, respectively. Lane L: 50-bp DNA ladders. Lane NC: ddH₂O.

FIG. 3 illustrates a comparison of the reaction time of NNV detection by using a conventional PCR machine and the microfluidic LAMP system. Lane L: 100-bp DNA ladders. Lanes 1-3 and lanes 4-6 are the results using the microfluidic LAMP system and a conventional LAMP, respectively. Lanes 1, 4 indicate RT-LAMP that is carried out for 75 min, lanes 2, 5 and lanes 3, 6 indicate RT-LAMP that is carried out for 60 min and 45 min, respectively.

FIG. 4 illustrates sensitivity comparison of RT-LAMP and RT-PCR assays. (a) RT-PCR products; (b) RT-LAMP products. Lane L: 50-bp DNA ladders, lane NC: negative control using ddH₂O, lanes 1-8: 10⁻¹ dilutions of 10-ng cDNA from the NNV RNA1.

FIG. 5 illustrates specificity of the RT-LAMP assay for NNV infected grouper and non-NNV test samples (lane L: 50-bp DNA ladders, lane NC: negative control used by ddH₂O, lane 1: RNA from NNV infected grouper, lanes 2-8 indicate tested samples are from Dengue virus, HBV, influenza A virus, E. coli; Staphylococcus aureus; Vibrio spp. and human A549 cells, respectively. 10 ng DNA/RNA is used for RT-LAMP).

### DETAILED DESCRIPTION OF THE INVENTION

As utilized in accordance with the present disclosure, the following terms, unless otherwise indicated, shall be understood to have the following meanings:

The term “oligonucleotide” as referred to herein means single-stranded or double-stranded nucleic acid polymers of at least 10 bases in length. In certain embodiments, the nucleotides comprising the oligonucleotide can be ribonucleotides or deoxyribonucleotides or a modified form of either type of nucleotide. Said modifications include base modifications such as bromuridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “oligonucleotide” specifically includes single and double stranded forms of DNA.

Often, ranges are expressed herein as from “about” one particular value and/or to “about” another particular value. When such a range is expressed, an embodiment includes the range from the one particular value and/or to the other particular value. Similarly, when values are expressed as approximations, by use of the word “about,” it will be understood that the particular value forms another embodiment. It will be further understood that the endpoints of each of the ranges are significant both in relation to and independently of the other endpoint.

It must be noted that, as used in the specification and the appended claims, the singular forms “a,” “an” and “the” include plural referents unless the context clearly dictates otherwise. Thus, unless otherwise required by context, singular terms shall include the plural and plural terms shall include the singular.

One subject of the invention is to provide a kit for rapidly detecting a target nucleic acid fragment, the target nucleic acid fragment comprising a purification recognized fragment and an amplification specific fragment, which kit comprises:

a magnetic bead linked to an oligonucleotide being able to hybridize to the purification recognized fragment;

an inner primer pair and an outer primer pair being specific to the amplification specific fragment and suitable for loop-mediated isothermal amplification; and

reagents for loop-mediated isothermal amplification.

According to the invention, the target nucleic acid fragment is preferably a pathogen-specific nucleic acid fragment or an immune-related gene-derived nucleic acid fragment that can be used to detect a pathogen. In one preferred embodiment of the invention, the target nucleic acid fragment is selected from the group consisting of a nervous necrosis virus (NNV)-specific fragment, an Iridovirus-specific fragment, a Vibrio-specific fragment and an immune-specific Mx of grouper. More preferably, the target nucleic acid fragment is a nervous necrosis virus-specific fragment. NNV comprises two single-stranded RNA (ssRNA), RNA1 and RNA2, which codes RNA-dependent RNA polymerase and a major coat protein, respectively (Mori et al, 1992, Virology 187, 368-371; Nishizawa et al. 1997, Appl. Environ. Microbiol. 63, 1633-1636). RNA1 and RNA2 are able to be used as the target nucleic acid fragment according to the invention.

According to the invention, the purification recognized fragment is a specific fragment in the target nucleic acid fragment. The purification recognized fragment can be used for purification and distinguished from other nucleic acid fragments in a sample. Artisans skilled in the field of the invention can obtain the purification recognized fragment with the use of a relevant commercial sequence analysis software.

According to the invention, the amplification specific fragment is a specific fragment in the target nucleic acid fragment. The amplification specific fragment can be distinguished from other nucleic acid fragments in a sample after amplification. Artisans skilled in the field of the invention can obtain the amplification specific fragment with the use of a relevant commercial sequence analysis software.

In one preferred embodiment of the invention, the distance between the purification recognized fragment and the amplification specific fragment is from about 200 bp to about 500 bp. Such range facilitates the loop-mediated isothermal amplification after purification, and the efficiency thereof is better.

According to the invention, the magnetic bead and the oligonucleotide being able to hybridize to the purification recognized fragment linked thereon are designed for purifying the target nucleic acid fragment in the sample to facilitate the following operations. Artisans skilled in the field of the invention can choose an appropriate material and size of the magnetic bead. In one preferred embodiment of the invention, the diameter of the magnetic bead is from about 1 μm to about 5.0 μm. Such range both facilitates the linkage between the magnetic bead and the oligonucleotide being able to hybridize to the purification recognized fragment and the following operations.

According to the invention, the manner for linking the oligonucleotide being able to hybridize to the purification recognized fragment to the magnetic bead is well-known to artisans skilled in the field of the invention according to the disclosure of the specification of the invention. A conventional manner of linking an oligonucleotide to a magnetic bead can be applied in the invention. In one preferred embodiment of the invention, the magnetic bead is linked to the oligonucleotide being able to hybridize to the purification recognized fragment through an amide bond or a carboxylate bond.

According to the invention, the oligonucleotide being able to hybridize to the purification recognized fragment is designed for hybridizing the purification recognized fragment of the target nucleic acid through a complementary feature. The hybridizing complex is further purified by a magnetic force. Moreover, the two hybridized strands can be separated in the subsequently loop-mediated isothermal amplification, and further amplified.

According to the invention, the length and sequence of the oligonucleotide being able to hybridize to the purification recognized fragment are designed according to the purification recognized fragment. Artisans skilled in the field of the invention can design the purification recognized fragment. In one preferred embodiment of the invention, the length of the oligonucleotide being able to hybridize to the purification recognized fragment is from about 20 bp to about 40 bp.

According to the invention, the inner primer pair and an outer primer pair being specific to the amplification specific fragment and suitable for loop-mediated isothermal amplification and the reagents for loop-mediated isothermal amplification are well-known to artisans skilled in the field of the invention according to the disclosure of the specification of the invention. For example, the design described in Notomi et al, 2000, Nucleic Acids Res. 28, e63. Such disclosure is incorporated herein by reference.

In one preferred embodiment of the invention, the target nucleic acid is a RNA fragment, and the kit further comprises reagents for reverse transcription polymerase chain reaction. It allows carrying on a reverse transcription polymerase chain reaction before the purification and amplification.

In one preferred embodiment of the invention, the kit further comprises a microfluidic chip. As used herein, “a microfluidic chip” refers to an apparatus that detection-required elements such as a sample loading chamber, a pneumatic micro-pump, a reaction chamber, a micro-valve, and a waste chamber, are integrated thereon. The sample or reagents are driven to move in micro-channels connecting the elements by electroosmotic flow generated by voltage applied or the use of micro-pumps or a centrifugal force to complete the reaction. The microfluidic chip also known as a lab-on-a-chip, and the use of microfluidic chip for biomedical detection or analysis has advantages of reduced manual error, increased system stability, reduced energy consumption and reduced amount of samples, reduced the capacity and time-saving.

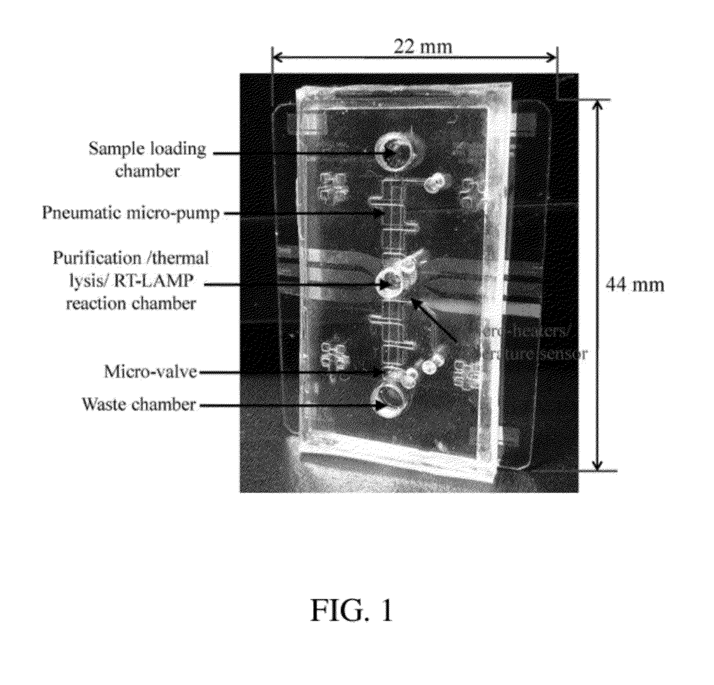

In one preferred embodiment of the invention, the microfluidic chip comprises a microfluidic control module and an isothermal amplification module is shown in FIG. 1. The microfluidic control module comprises one glass substrate with metallization patterns and two polydimethylsiloxane (PDMS) layers, namely a thick PDMS layer with structures for the microfluidic channel and a thin-film PDMS membrane for the air chambers. The microfluidic control module further comprises one sample loading chamber, one purification/thermal lysis/LAMP reaction chamber, a waste chamber and two sets of pneumatic micro-pumps with normally-closed micro-valves. These valves are designed for liquid delivery and to prevent backflow in the miniature system. The optimal design parameters, microfabrication and characterizations for the module can be referenced of Yang et al., 2009 (Yang et al., 2009, Microfluid. Nanofluid. 6, 823-833). Such disclosure is incorporated herein by reference.

The isothermal amplification module of the microfluidic chip preferably comprises two sets of self-compensated, array-type micro-heaters and a temperature sensor is built to generate the temperature distribution with a high thermal uniformity within the thermal lysis/LAMP reaction chamber. Without using additional control circuits, the isothermal amplification module is fabricated with surrounding heating grids which are used as compensating heaters for the edge areas. Hence, the amplification efficiency of the LAMP process can be enhanced within the reaction chamber distributed with a high thermal uniformity. Details of the self-compensated, isothermal amplification module and the microfabrication process can be found in the previous literature (Hsieh et al., 2009, Microfluid. Nanofluid. 6, 797-809). Meanwhile, in one preferred embodiment of the invention, an application specific integrated circuit (ASIC) controller is used to control all the components including the microfluidic control module and the isothermal amplification module. A heat sink with a pocket for placement of a permanent magnet and an adjustable magnetic stage directly connected to a compressed gas tank regulated by the EMV are employed. The permanent magnet on the magnetic stage can be engaged and slided into the pocket automatically during the purification process by providing a digital signal into the EMV, followed by disengaging it from the pocket during the re-suspension and LAMP processes. Thus, the sample transportation process and the temperature field distribution can be precisely and automatically controlled.

Preferably, the kit according to the invention further comprises an apparatus or a system for detecting the product of the amplification. In one preferred embodiment of the invention, the kit further comprises a gel electrophoresis system or an absorbance detection system for detecting a product of loop-mediated isothermal amplification.

In one preferred embodiment of the invention, in the amplification processes within LAMP, pyrophosphate is released, followed with nucleic acid elongation, and is reacted with magnesium ions to cause a change in turbidity in the mixture. Consequently, an optical system is integrated in the future to sense the turbidity variation for the amount of end product.

In one preferred embodiment of the invention, the kit further comprises a lysis buffer for lysing the sample. More preferably, the lysis buffer is able to preliminarily lysing the sample to facilitate the following purification and amplification.

Another subject of the invention is to provide a kit for detecting a pathogen in fish by detecting a pathogen target nucleic acid fragment in a sample, which kit comprises the kit for rapidly detecting a target nucleic acid fragment mentioned above.

Still another subject of the invention is to provide a method for rapidly detecting a target nucleic acid fragment, the target nucleic acid fragment comprising a purification recognized fragment and an amplification specific fragment, which method comprises:

(a) purifying a nucleotide with a magnetic bead, wherein the magnetic bead linked to an oligonucleotide being able to hybridize to the purification recognized fragment;

(b) conducting a loop-mediated isothermal amplification with an inner primer pair and an outer primer pair being specific to the amplification specific fragment and the nucleotide purified in step (a), wherein the inner primer pair and the outer primer pair are suitable for loop-mediated isothermal amplification; and

(c) detecting a product of the loop-mediated isothermal amplification.

Still another subject of the invention is to provide a method for detecting a pathogen in fish by detecting a pathogen target nucleic acid fragment in a sample, which method comprises the method for rapidly detecting a target nucleic acid fragment mentioned above.

In one preferred embodiment of the invention, the method according to the invention is applied to detect if a grouper infected by a nervous necrosis virus, and the method allows rapid detection of nervous necrosis virus in an aquacultured grouper by purification of RNA of nervous necrosis virus from the fish tissue samples. The magnetic bead and oligonucleotide being able to hybridize to the purification recognized fragment are used as a probe to specifically detect the target RNA sample dissolved in the whole-tissue lysate, and also to hybrid on the surface of the magnetic bead. Hereafter, the combination of the built-in microfluidic control module and permanent magnet is applied to purify the magnetic complex from the sample. In addition, the one-step isothermal RT-LAMP is performed to amplify the target gene by the use of the isothermal amplification module of the chip. Thus, the kit and method according to the present invention provides an automated platform of a fast diagnosis of disease in aquaculture with little need of human intervention.

In order to simplify the process of RNA extraction, the method according to the present invention uses a sequence-specific probe linked to the magnetic bead to provide a rapid and sensitive detection of nucleic acids. In order to decrease the operating time, the first incubation step can be shortened to 20 min or 10 min without affecting the final result from the RT-LAMP assay. With this combination of the RNA captured beads and one-step RT-LAMP, the entire analysis protocol can be completed within 1 h.

The RT-LAMP assay has been shown to be a rapid, sensitive and specific method for detecting the NNV in grouper tissues. In the embodiments of the invention, a new microfluidic LAMP system integrated with two functional modules utilizing magnetic beads has been demonstrated for rapid RNA extraction and for reverse transcription-isothermal amplification. This developed microfluidic LAMP system provides a useful tool for rapid, routine diagnosis and for large-scale screening in field studies.

The following Examples are given for the purpose of illustration only and are not intended to limit the scope of the present invention.

### Example

### Materials and Methods

Experimental Procedures

The microfluidic chip shown in FIG. 1 is applied in the example. RNA1 of the NNV from the infected grouper is isolated by utilizing probe-conjugated magnetic beads, followed by performing the one-step RT and isothermal amplification with the incorporation of built-in micro-heaters and temperature sensors. Briefly, a random sampling of the grouper larvae is first carried out in the fishery, followed by a grinding process with a pestle in an 1.5 mL microcentrifuge tube. The on-chip micro-heaters are then activated to perform the thermal lysis of the bio-samples when the tissue fluid of the grouper is loaded into a purification chamber of the microfluidic chip.

Then, the hybridization of the released RNA1 of the NNV is performed by loading the RNA1-specific, probe-conjugated, magnetic beads into the purification chamber at room temperature. Next, a permanent magnet is attached underneath the chip to attract the hybridized RNA1-probe-conjugated magnetic complexes onto the surface of the purification chamber, followed by flowing a washing buffer through the purification chamber continuously using an integrated micro-pump. All the other unbound interferents in the biological solution would then be washed away into a waste chamber. The one-step RT-LAMP reagent is then loaded into a sample loading chamber, followed by transporting it into the purification chamber to perform the subsequent synthesization of cDNA and isothermal amplification simultaneously. Significantly, a reverse transcriptase with a high thermal stability is employed in the assay, allowing for concurrent reactions including cDNA synthesization from ssRNA and for the isothermal amplification of target genes. With this approach, the target viral RNA can be isolated from the biological tissues and then are used in the subsequent identification of genetic patterns associated with aquaculture diseases.

Detailed procedure is illustrated below.

Infectious Fish Samples Preparation

The NNV infected grouper are first sampled randomly and collected from cultivation farms in Qigu and Yong'an, Taiwan. All the fish samples, including the brain and other tissues, were stored at −80° C. prior to the viral RNA extraction process and the on-chip analysis. To avoid the clogging of the micro channels by large or tough fish tissue, the fish organs were grinded using a tissue grinder to obtain virus particles from extracted samples.

RNA Probe and Primers Design

In order to purify the target RNA1 of the NNV from grouper tissue samples, a RNA1-specific probe has been designed and utilized to target the NNV RNA-dependent RNA polymerase (GeneBank accession no. AY721616). In addition, the specificity of the RNA-probe-conjugated magnetic beads was verified by performing a conventional RT-PCR process with the purified RNA samples and two sets of primer pairs including the NNV RNA1 and NNV RNA2 primer sets, which were designed from accession nos. AY721616 and AY721615, respectively, from the National Center for Biotechnology Information (NCBI, USA) GeneBank. Furthermore, two sets of primers including the outer primer pair (NNV-F3/NNV-B3) and the inner primer pair (NNV-FIP/NNV-BIP) were also developed for the LAMP process. All the specific primer sets were designed using the Primer Explorer Software (http://primerexplorer.jp/elamp4.0.0/index). Details of the primer sets and the probe sequences can be found in Table 1.

TABLE 1

| | | | | | SEQ |
|---|---|---|---|---|---|
| | | | | GeneBank | ID |
| Primer | Location | Length | Sequence | ref. | NO. |
| NNV-F3 | 521→539 | 19 nt | 5′ | AY721616 | 1 |
| | | | ACGTGGACATGCA | | |
| | | | TGAGTT 3′ | | |
| NNV-B3 | 761→743 | 19 nt | 5′ | AY721616 | 2 |
| | | | TCACGCAGGATCT | | |
| | | | GCATCA 3′ | | |
| NNV-FIP | 647→627/ | 46 nt | 5′ | AY721616 | 3 |
| | TTTT/ | | CGGTAGTGAACGG | | |
| | 582→601 | | AGTCGTCAGTTTT | | |
| | | | AAGTACTGTGTCC | | |
| | | | GGAGAGG 3′ | | |
| NNV-BIP | 660→679/ | 42 nt | 5′ | AY721616 | 4 |
| | TTTT/ | | GAAGGATGTGCGC | | |
| | 731→714 | | CATCGCATTTTAA | | |
| | | | ACCACGAGGTCGG | | |
| | | | GAG 3′ | | |
| NNV | TTTTTTTTT | 31 nt | 5′ | AY721616 | 5 |
| probe | T/ | | TTTTTTTTTTACCG | | |
| | 287→267 | | AGATACGCACTAG | | |
| | | | CTCC 3′ | | |

Magnetic Bead-Based RNA1 Extraction and Hybridization

A specific NNV RNA1-probe is conjugated onto the surface of the magnetic beads (MAGBEAD AGT-003-05, Applied gene technologies technologies, USA) by utilizing the carboxylated linkage prior to the on-chip analysis (Hawkins et al., 1994, Nucleic Acids Res. 22, 4543-4544). A grinding process is first performed by using a pestle with a 200 μL of lysis buffer [62.5 mM Tris, pH8.3, 95 mM KCl, 3.8 mM MgCl₂, 12.5 mM dithiothreitol (DTT), and 0.63% octyl phenoxylpoly ethoxylethanol (NP-40)] in an 1.5 mL microcentrifuge tube to collect the whole tissue lysates. Then, 50 μL of whole tissue lysates is then loaded into the purification chamber, where the RNA1-specific probe-conjugated magnetic beads with a volume of 5 μL are pre-loaded, to perform the thermal lysis process of the virus at 95° C. for 5 min. After that, a temperature field of 60° C. for 15 min is generated within the purification chamber for the hybridization process between the target RNA1 of the NNV and the specific probe-conjugated magnetic beads. The temperature (from 58° C. to 65° C.) and reaction time (from 10 min to 45 min) for hybridization have been optimized for operating temperature and reaction time. Then, a magnetic field (˜300 Gauss) generated by a permanent magnet is used to concentrate and to collect the target RNA-bound magnetic complexes onto the surface of the purification chamber, followed by washing all the other biological substances away into the waste chamber with the incorporation of micro-pumps and micro-valves. Next, the purified magnetic complexes are re-suspended into double distilled water (ddH₂O) with a volume of 50 μL. Note that only 10 μL of RNA-bound magnetic complexes are used for the subsequent one-step RT-LAMP process. Alternatively, the purified RNA can be also stored for further biomedical applications by extracting RNA-bound magnetic complexes from the purification chamber using pipettes.

One-Step RT-LAMP

A final reaction volume of 30 μL is employed for the one-step RT-LAMP process and the LAMP reaction is modified as previously described (Notomi et al., 2000, Nucleic Acids Res. 28, e63). The reaction mixture comprising 10 μL of target RNA-bound magnetic complexes, 60 μM of NNV-FIP and NNV-BIP primers, 10 μM of NNV-F3 and NNV-B3 primers, 3 μL of 10×Bst polymerase reaction buffer and 1 μL of Bst DNA polymerase large fragment (8 U/μL, NEW ENGLAND Bio-Lab Inc., USA) and 0.5 μL of ThermoScript™ RNase H⁻ reverse transcriptase (15 U/μL, Invitrogen, USA) is loaded into the LAMP reaction chamber to perform the cDNA synthesization and isothermal amplification simultaneously. The reaction is terminated by heating the samples at 80° C. for 2 min, followed by synthesizing the cDNA from RNA1 and amplifying the target region of the cDNA at 63° C. for 45 min. The optimization of the amplification efficiency for the RT-LAMP assay is also carried out by verifying two major reaction conditions, specifically, the reaction temperature (from 57° C. to 67° C.) and the reaction time (from 30 min to 90 min). The RT-LAMP products are analyzed by slab-electrophoresis technique in a 2% agarose gel.

Results and Discussion

Characterization of the Microfluidic System

A microfluidic control module comprising two sets of pneumatic micro-pumps with three PDMS membranes and a floating block structure is used to precisely transport bio-samples and to prevent backflow. The time-phased deformation of successive PDMS membranes underneath the microchannel generates a peristaltic effect that drives the liquid along the microchannel when the compressed air fills up the interconnected air chambers sequentially. Two essential parameters including the driving frequency (f_{d}) of the electromagnetic valve (EMV) and the applied compressed air pressure can be used to control the flow pumping rate for sample transport. EMVs (SMC Inc., S070M-5BG-32, Japan) are used to control the micro-pumps by regulating the operating frequency of the EMVs such that the thin-film PDMS membranes are deflected under the supplied compressed air pressure. These parameters have been optimized from characterizations of the micro-pump flow rate in Yang et al., 2009 (Yang et al., 2009, Microfluid. Nanofluid. 6, 823-833). The maximum flow rate has been measured to be 900 μL/min at f_{d}=90 Hz at an air pressure of 20 psi. In addition, the floating block structure of the normally-closed micro valve capable of increasing the flow pumping rate has also been demonstrated to block the liquid sample in the microchannel successfully. The thin-film PDMS membrane and another air chamber placed underneath the floating block structure allow the deflection of the PDMS membrane so that the fluid can flow through the valve structure in the microchannel. Note that the floating block structure is not bonded with the thin-film PDMS membrane and can be used to prevent the backflow generation such that the fluid can flow in only one direction. A back pressure as high as 8509 N/m² can be generated at f_{d}=90 Hz and an air pressure of 20 psi. Hence, the microfluidic control module is capable of transporting the samples/reagents or blocking the fluids inside the microfluidic channel.

In addition to the microfluidic control module, a self-compensated, array-type isothermal amplification module comprised of two sets of micro-heaters and a temperature sensor is also integrated for amplification of target genes. Despite the high temperature ramping rate of the micro-heaters, the thermal uniformity within the reaction chamber is also an important factor during the RT-LAMP process. The temperature inside the reaction chamber is found to be uniformly distributed with a variation of less than 0.5° C. at the set point (Hsieh et al., 2009, Microfluid. Nanofluid. 6, 797-809). Hence, the uniform temperature distribution of this prototype microfluidic LAMP system enables the isothermal amplification to be completed with a highly amplification efficiency.

Optimization of RNA-Probe Hybridization and the One-Step RT-LAMP Process

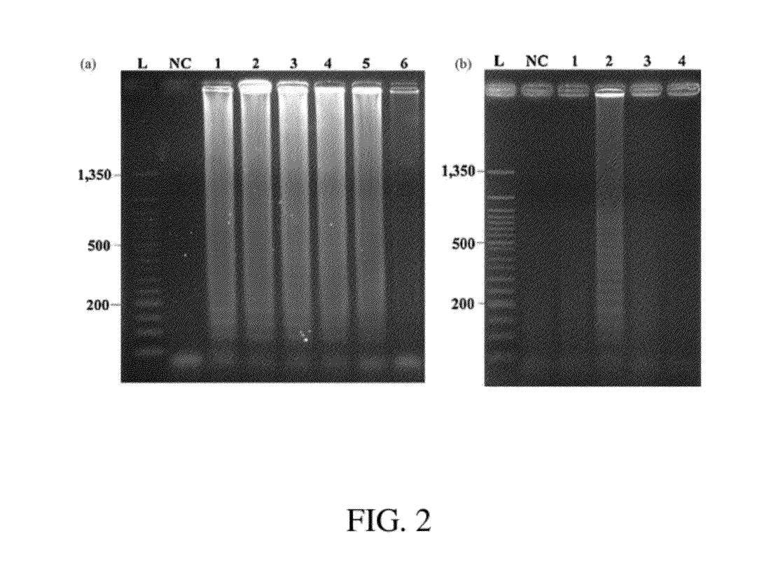

The operating conditions for the microfluidic LAMP system for rapid viral RNA extraction and for the one-step RT-LAMP process have been optimized by testing five key experimental factors, namely (1) the reaction temperature during the one-step RT-LAMP process, (2) the reaction time for the one-step RT-LAMP process, (3) specificity of the RNA-probe-conjugated magnetic beads, (4) the reaction temperature for RNA hybridization, and (5) the reaction time for the RNA hybridization process. The experimental verification of these factors during the one-step RT-LAMP process is first carried out by utilizing a conventional thermo-block PCR machine (MyCycler™ thermal cycler, BioRad, USA) and is shown in FIG. 2. FIG. 2(a) shows the experimental results from the one-step RT-LAMP assay with different reaction temperatures. Reaction temperatures ranging from 57° C. (lane 1) to 67° C. (lane 6) are tested.

The target genes of the NNV RNA1 are found to have been successfully amplified with an isothermal temperature condition between 57° C. (lane 1) and 65° C. (lane 5). The results show that the optimal reaction temperature distribution of 61° C. can be employed during the one-step RT-LAMP process. The reaction time of the LAMP process is also considered as a major experimental parameter for the one-step RT-LAMP assay. Experimental data shows that target genes can also be amplified in approximately 60 min by using the thermo-block PCR machine. Moreover, the target RNA1 of the NNV in the tissue samples can be rapidly purified and extracted by utilizing the hybridization with the RNA1-specific probe-conjugated magnetic beads. Similarly, hybridization at different temperature conditions of 58° C. (lane 1), 60° C. (lane 2), 63° C. (lane 3) and 65° C. (lane 4) have been tested and are shown in FIG. 2(b), followed by performing the standard one-step RT-PCR process. It is found that the NNV RNA1 has been successfully hybridized and amplified at a temperature of 60° C. (lane 2). Furthermore, the reaction time for hybridization has also been tested and the experimental results show that target the RNA1 of the NNV can also be conjugated onto the surface of the magnetic beads in approximately 15 min. However, in order to coordinate the temperature field during the entire diagnostic process, the reaction chamber was set at a temperature of 60° C. to complete the entire assay including the RNA hybridization and RT-LAMP process. Consequently, the proposed RNA purification and one-step RT-LAMP assay can be completed using the following three optimized steps: (1) thermal lysis at 95° C. for 5 min, (2) RNA hybridization at 60° C. for 15 min, and (3) one-step RT-LAMP at 60° C. for 60 min.

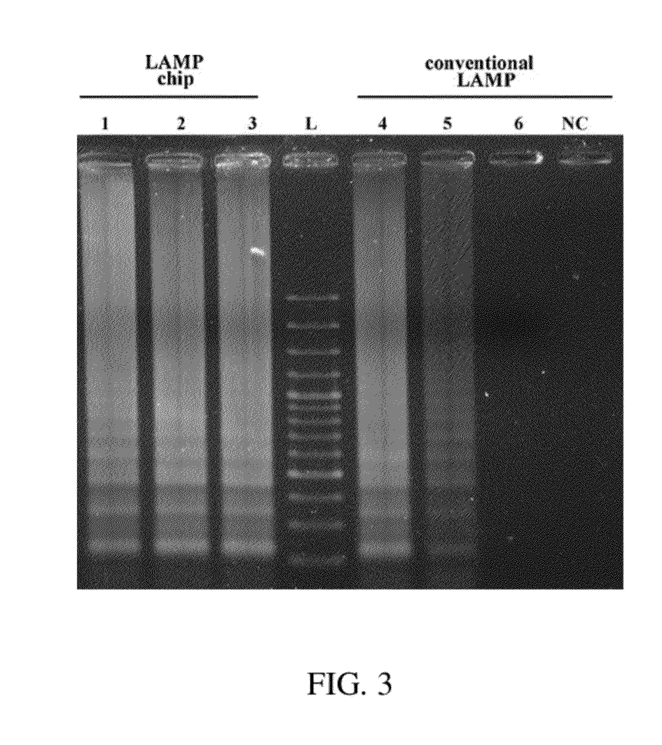

The developed protocol for RNA purification and isothermal amplification has also been carried out in the integrated microfluidic LAMP system in an automatic manner. Note that the composition of the one-step RT-LAMP reagents are the same as the one described previously, except it is proportionally decreased by half with a final volume of 15 μL when used in the microfluidic system. FIG. 3 shows a comparison of reaction times between the conventional one-step RT-LAMP performed by the thermo-block PCR machine and the microfluidic LAMP system. Successful amplification is observed in lane 3 (45 min at 60° C.) by the microfluidic LAMP system, indicating that isothermal amplification can be completed in a shorter period of time by utilizing the miniature system. Significantly, the experimental data also reveals that a highly amplification efficiency can be completed due to the uniformly-distributed temperature field generated by the microfluidic LAMP system with its self-compensated temperature control module.

Consequently, a one-step RT-LAMP process with high amplification efficiency can be achieved in a shorter period of time.

Sensitivity

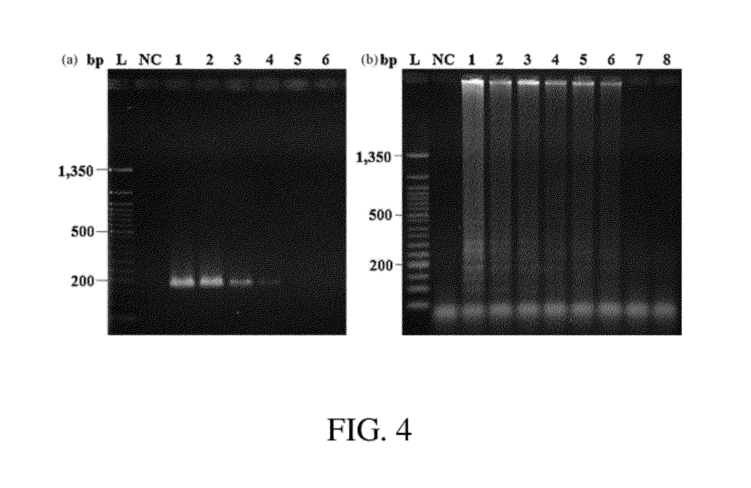

The sensitivity of the developed RT-LAMP protocol is also explored, as shown in FIG. 4. The 10 ng of NNV cDNA sample is prepared from 2 μg of extracted total RNA, 2 μM of random hexamers, 0.4 μM of dNTPs, 5 μL of 5 reverse transcriptase working buffer and 200 U of MMLV reverse transcriptase (Promega, USA) at 42° C. with a 60 min incubation. These tested cDNA samples are used to perform a 10-fold serial dilution and are tested in both the conventional RT-PCR process (FIG. 4(a)) and the one-step RT-LAMP assay (FIG. 4(b)).

The detection limit of the developed system is found in lane 4 in FIG. 4(a) and lane 7 in FIG. 4(b). Namely, 1-10 pg of cDNA for the conventional RT-PCR process and 10-100 fg of cDNA for the one-step RT-LAMP assay are detected successfully, respectively. The high sensitivity of the proposed one-step RT-LAMP protocol is verified when compared with the traditional one-step RT-PCR protocol.

Specificity

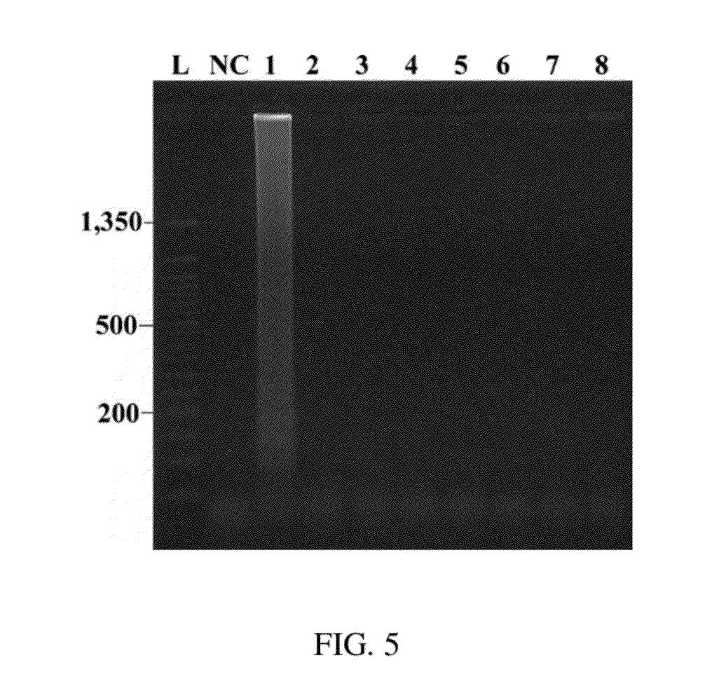

A high specificity is one of the major advantages of the invention, since only target RNA would be bound onto the surface of the probe-conjugated magnetic beads. The specificity of the system is then verified by utilizing different total DNA/RNA samples extracted from the NNV infected grouper, Dengue virus, Hepatitis B virus, influenza A virus, E. coli; Staphylococcus aureus; Vibrio spp. And human lung cancer A549 cells. Note that the DNA/RNA template is extracted by thermal lysis at 95° C. and each of the extracted 10 ng samples is employed in the NNV-LAMP detection. The high specificity of the NNV infected grouper, isolated from different cultivation farms, can be achieved by utilizing the one-step RT-LAMP assay. The specificity of the reaction for NNV-RT-LAMP is also determined by performing a cross-reactivity assay with the aforementioned different sources of RNA/DNA (FIG. 5). It is clearly observed from the results that only the sample extracted from the NNV infected grouper is amplified successfully. The experimental data, therefore, demonstrates a high specificity for the proposed one-step RT-LAMP assay since the target RNA is captured and separated successfully from the clinical sample by utilizing the magnetic beads with a specific probe.

While embodiments of the present invention have been illustrated and described, various modifications and improvements can be made by persons skilled in the art. The present invention is not limited to the particular forms as illustrated, and that all the modifications not departing from the spirit and scope of the present invention are within the scope as defined in the appended claims.

What is claimed is:

1. A kit for rapidly detecting a target nucleic acid fragment, the target nucleic acid fragment comprising a purification recognized fragment and an amplification specific fragment, the kit comprising:
a magnetic bead linked to an oligonucleotide being able to hybridize to the purification recognized fragment;
an inner primer pair and an outer primer pair being specific to the amplification specific fragment and suitable for loop-mediated isothermal amplification; and
reagents for loop-mediated isothermal amplification.

2. The kit according to claim 1, wherein the diameter of the magnetic bead is from about 1 μm to about 5.0 μm.

3. The kit according to claim 1, wherein the magnetic bead is linked to the oligonucleotide being able to hybridize to the purification recognized fragment through an amide bond or a carboxylate bond.

4. The kit according to claim 1, wherein the length of the oligonucleotide being able to hybridize to the purification recognized fragment is from about 20 bp to about 40 bp.

5. The kit according to claim 1, wherein in the target nucleic acid fragment, the distance between the purification recognized fragment and the amplification specific fragment is from about 200 bp to about 500 bp.

6. The kit according to claim 1, further comprising reagents for reverse transcription polymerase chain reaction.

7. The kit according to claim 1, further comprising a microfluidic chip.

8. The kit according to claim 7, wherein the microfluidic chip comprises a microfluidic control module and an isothermal amplification module, wherein
the microfluidic control module comprises:
a glass substrate comprising metallization patterns, a thick polydimethylsiloxane (PDMS) layer and a thin-film PDMS membrane; wherein the thick polydimethylsiloxane layer comprises a microfluidic channel and the thin-film PDMS membrane comprises a sample loading chamber, a reaction chamber, and a waste chamber; and
a pneumatic micro-pump with normally-closed micro-valves designed for liquid delivery and to prevent backflow; and
the isothermal amplification module comprises:
a self-compensated, array-type micro-heater and a temperature sensor to generate the temperature distribution with a high thermal uniformity within the reaction chamber.

9. The kit according to claim 8, wherein the microfluidic chip comprises an application specific integrated circuit (ASIC) controller to control the microfluidic control module and the isothermal amplification module.

10. The kit according to claim 9, wherein the application specific integrated circuit controller comprises a heat sink with a pocket for placement of a permanent magnet and an adjustable magnetic stage directly connected to a compressed gas tank regulated by a electromagnetic valve (EMV), the permanent magnet on the magnetic stage is able to be engaged and slided into the pocket automatically by providing a digital signal into the EMV, followed by disengaging it from the pocket.

11. The kit according to claim 1, further comprising a gel electrophoresis system or an absorbance detection system for detecting a product of loop-mediated isothermal amplification.

12. The kit according to claim 1, wherein the target nucleic acid fragment is a pathogen-specific nucleic acid fragment or an immune-related gene-derived nucleic acid fragment.

13. The kit according to claim 1, wherein the target nucleic acid fragment is selected from the group consisting of a nervous necrosis virus-specific fragment, an Iridovirus-specific fragment, a Vibrio-specific fragment and an immune-specific Mx of grouper.

14. A kit for detecting a pathogen in fish by detecting a pathogen target nucleic acid fragment in a sample, the kit comprising the kit for rapidly detecting a target nucleic acid fragment according to claim 1.

15. The kit according to claim 14, further comprising a lysis buffer for lysing the sample.

16. The kit according to claim 14, wherein the target nucleic acid fragment is a pathogen-specific nucleic acid fragment or an immune-related gene-derived nucleic acid fragment.

17. The kit according to claim 14, wherein the target nucleic acid fragment is selected from the group consisting of a nervous necrosis virus-specific fragment, an Iridovirus-specific fragment, a Vibrio-specific fragment and an immune-specific Mx of grouper.

18. A method for rapidly detecting a target nucleic acid fragment, the target nucleic acid fragment comprising a purification recognized fragment and an amplification specific fragment, the method comprising:
(a) purifying a nucleotide with a magnetic bead, wherein the magnetic bead linked to an oligonucleotide being able to hybridize to the purification recognized fragment;
(b) conducting a loop-mediated isothermal amplification with an inner primer pair and an outer primer pair being specific to the amplification specific fragment and the nucleotide purified in step (a), wherein the inner primer pair and the outer primer pair are suitable for loop-mediated isothermal amplification; and
(c) detecting a product of the loop-mediated isothermal amplification.

19. A method for detecting a pathogen in fish by detecting a pathogen target nucleic acid fragment in a sample, the method comprising the method for rapidly detecting a target nucleic acid fragment according to claim 18.

20. The method according to claim 19, further comprising lysing the sample with a lysis buffer before step (a).


SEQUENCE LISTING (5 sequences)

SEQ ID NO: 1 - DNA, 19 bp, artificial
  primer
acgtggacatgcatgagtt

SEQ ID NO: 2 - DNA, 19 bp, artificial
  primer
tcacgcaggatctgcatca

SEQ ID NO: 3 - DNA, 46 bp, artificial
  primer
cggtagtgaacggagtcgtcagttttaagtactgtgtccggagagg

SEQ ID NO: 4 - DNA, 42 bp, artificial
  primer
gaaggatgtgcgccatcgcattttaaaccacgaggtcgggag

SEQ ID NO: 5 - DNA, 31 bp, artificial
  probe
ttttttttttaccgagatacgcactagctcc